(19) United States
(12) Patent Application Publication
(10) Pub. No.: US 2016/0235710 A1
(43) Pub. Date: Aug. 18, 2016

(54) COMPOSITION FOR TREATING OR PREVENTING ERECTILE DYSFUNCTION COMPRISING LDD175 AS ACTIVE INGREDIENT

(71) Applicants: ANYGEN, INC., Jeollanam-do (KR); SAMSUNG LIFE PUBLIC WELFARE FOUNDATION., Seoul (KR); SNU R&D FOUNDATION, Seoul (KR); INDUSTRIAL COOPERATION FOUNDATION CHONBUK NATIONAL UNIVERSITY, Jeollabuk-do (KR)

(72) Inventors: Sung Won LEE, Seoul (KR); Jong Kwan PARK, Jeolabuk-do (KR); In Suk SO, Seoul (KR); Hyun Hwan SUNG, Seoul (KR); Jae Il KIM, Gwangju (KR); Young Chul KIM, Gwangju (KR); Jin Seok PARK, Gwangju (KR)

(21) Appl. No.: 14/994,382

(22) Filed: Jan. 13, 2016

(30) Foreign Application Priority Data
Jan. 14, 2015 (KR) 10-2015-0006598

(51) Int. Cl. A61K 31/407; A61K 31/519; A61K 9/00

(52) U.S. Cl. CPC A61K 31/407; A61K 9/0019; A61K 31/519

(57) ABSTRACT

The present invention relates to a pharmaceutical composition for treating or preventing erectile dysfunction, containing LDD175 as an active ingredient. Furthermore, the present invention relates to a co-administration preparation for treating or preventing erectile dysfunction, containing LDD175 and a PDE5 inhibitor as active ingredients. The LDD175 of the present invention has an excellent corporal smooth muscle relaxation effect, and thus significantly improved erectile function. Since the LDD175 of the present invention acts on BK_{Ca} channels, and thus is expected to have little adverse cardiovascular effects. The LDD175 of the present invention exhibits an erectile dysfunction therapeutic ability equivalent to that of udenafil, which is a PDE5 inhibitor, and showed a synergistic corporal smooth muscle relaxation effect when being co-administered with udenafil.

### CROSS-REFERENCE TO RELATED APPLICATIONS

This application claims the benefit and priority of Korean Patent Application No. 10-2015-0006598, filed Jan. 14, 2015. The entire disclosure of the above application is incorporated herein by reference.

### FIELD

The present disclosure relates to a novel use of 4-chloro-7-trifluromethyl-10H-benzo[4,5]furo[3,2-b]indole-1-carboxylic acid (LDD175) for medical utility, for example, for treating and preventing erectile dysfunction.

### BACKGROUND

Erectile dysfunction (ED) is a chronic disease defined as the consistent inability to initiate or maintain a sufficient erection for satisfactory sexual function (1993). ED is a common disease that affects 10 to 30 million men in the United States (Feldman et al., 1994). Currently oral phosphodiesterase type 5 (PDE5) inhibitors are the first-line treatment for patients since the introduction of sildenafil as an ED drug, which provide non-invasive, effective, and well-tolerated therapeutic options (Goldstein et al., 1998). However, the response rate is lower in sub-populations, including patients with diabetes mellitus, patients with severe vasculogenic ED, and post-radical prostatectomy patients (Hatzimouratidis and Hatzichristou, 2005; Eardley et al., 2010). These patients require more invasive treatment. Therefore, the development of novel therapeutic options is imperative for patients who are non-responsive to the PDE5 inhibitors.

BK_{Ca} channels are Ca²⁺-activated potassium channels, which are activated by an increase in concentration in intracellular Ca²⁺ or by the membrane depolarization with a large conductance (100-300 pS) (Marty, 1981). The BK_{Ca} channels play a very important role in the control of vascular smooth muscle tone under physiological conditions as well as in pathophysiological states, such as hypertension, stroke, atherosclerosis, diabetes, and ED (Hill et al., 2010). BK_{Ca} channel openers increase the efflux of potassium ions, leading to hyperpolarization, and thus decrease cell excitability and/or cause smooth muscle relaxation (Ghatta et al., 2006). Various types of BK_{Ca} channel openers, such as NS004 (Biagi et al., 2004), NS1619 (Olesen et al., 1994), mefenamic and flufenamic acids (Ottolia and Toro, 1994), NS1608 (Siemer et al., 2000), and the quinolinone analog (Hewawasam et al., 2003), have been known. However, the major limitations of this class of compounds are weak potency and insufficient in selectivity. Recently, Gormemis et al. optimized the pharmacophore groups of the benzofuroindole analogs (Gormemis et al., 2005). They found that 4-chloro-7-trifluoromethyl-10H-benzo[4,5]furo[3,2-b]indole-1-carboxylic acid (LDD175) was the most potent and effective BK_{Ca} channel activator (Gormemis et al., 2005). This novel BK_{Ca} channel opener was verified to have a relaxant activity on the urinary bladder (Dela Pena et al., 2009b), ileum (Dela Pena et al., 2009a), and uterus (Ahn et al., 2011) in a dose-dependent manner by activating BK_{Ca} channels. In addition, LDD175 has been reported to have no significant adverse cardiovascular effects in in vivo animal experiments (Dela Pena et al., 2009b).

Throughout the entire specification, many papers and patent documents are referenced and their citations are represented. The disclosures of cited papers and patent documents are entirely incorporated by reference into the present specification, and the level of the technical field within which the present invention falls and details of the present invention are explained more clearly.

### PATENT DOCUMENT

Korean Patent Publication No. 2011-0050680

### NON-PATENT DOCUMENTS

Consensus development conference statement. National Institutes of Health. Impotence. Dec. 7-9, 1992. Int J Impot Res 5: 181-284.
Ahn H S, Dela Pena I, Kim Y C, Cheong J H (2011). Pharmacology 87: 331-340.
Biagi G, Giorgi I, Livi O, Nardi A, Calderone V, Martelli A et al. (2004). V. Eur J Med Chem 39: 491-498.
Brink P R, Ramanan S V, Christ G J (1996). Am J Physiol 271: C321-331.
Dela Pena I C, Yoon S Y, Kim S M, Lee G S, Park C S, Kim Y C et al. (2009a). Arch Pharm Res 32: 413-420.
Dela Pena I C, Yoon S Y, Kim S M, Lee G S, Ryu J H, Park C S et al. (2009b). Pharmacology 83: 367-378.
Eardley I, Donatucci C, Corbin J, El-Meliegy A, Hatzimouratidis K, McVary K et al. (2010). J Sex Med 7: 524-540.
Edwards G, Henshaw M, Miller M, Weston A H (1991). Br J Pharmacol 102: 679-686.
Fabiyi A C, Gopalakrishnan M, Lynch J J, 3rd, Brioni J D, Coghlan M J, Brune M E (2003). BJU Int 91: 284-290.
Feldman H A, Goldstein I, Hatzichristou D G, Krane R J, McKinlay J B (1994). J Clin Epidemiol 47: 457-467.
Fukami Y, Toki Y, Numaguchi Y, Nakashima Y, Mukawa H, Matsui H et al. (1998). Life Sci 63: 1047-1055.
Ghatta S, Nimmagadda D, Xu X, O'Rourke S T (2006). Pharmacol Ther 110: 103-116.
Goldstein I, Lue T F, Padma-Nathan H, Rosen R C, Steers W D, Wicker P A (1998). N Engl J Med 338: 1397-1404.
Gonzalez-Corrochano R, La Fuente J, Cuevas P, Fernandez A, Chen M, Saenz de Tejada I et al. (2013). Br J Pharmacol 169: 449-461.
Gormemis A E, Ha T S, Im I, Jung K Y, Lee J Y, Park C S et al. (2005). Chembiochem 6: 1745-1748.
Hatzimouratidis K, Hatzichristou D G (2005). Drugs 65: 1621-1650.
Hewawasam P, Fan W, Ding M, Flint K, Cook D, Goggins G D et al. (2003). J Med Chem 46: 2819-2822.
Hill M A, Yang Y, Ella S R, Davis M J, Braun A P (2010). FEBS Lett 584: 2033-2042.
Kun A, Matchkov V V, Stankevicius E, Nardi A, Hughes A D, Kirkeby H J et al. (2009). Br J Pharmacol 158: 1465-1476.
Lee S W, Kang T M (2001). Urol Res 29: 359-365.
Lee S W, Wang H Z, Zhao W, Ney P, Brink P R, Christ G J (1999). Int J Impot Res 11: 189-199.
Marty A (1981). Nature 291: 497-500.
Olesen S P, Munch E, Moldt P, Drejer J (1994). Eur J Pharmacol 251: 53-59.
Ottolia M, Toro L (1994). Biophys J 67: 2272-2279.
Palmer L S, Valcic M, Melman A, Giraldi A, Wagner G, Christ G J (1994). J Urol 152: 1308-1314.
Rehman J, Chenven E, Brink P, Peterson B, Walcott B, Wen Y P et al. (1997). Am J Physiol 272: H1960-1971.
Siemer C, Bushfield M, Newgreen D, Grissmer S (2000). J Membr Biol 173: 57-66.
Sung H H, Chae M R, So I, Jeon J H, Park J K, Lee S W (2011). Int J Impot Res 23: 193-199.
Zhao W, Christ G J (1995). J Urol 154: 1571-1579.

### SUMMARY

### Technical Problem

The present inventors have searched for and endeavored to develop safe and effective erectile dysfunction (ED) drug candidates. As a result, the present inventors verified that LDD175, which is a BK_{Ca} channel opener, showed a relaxation effect of corporal smooth muscle cells through BK_{Ca} channel activation, and significantly improved penile erection, and thus have completed the present invention.

Therefore, in an embodiment, the present disclosure provides a composition for treating or preventing erectile dysfunction, containing LDD175 as an active ingredient.

In another embodiment, the present disclosure provides a co-administration preparation for treating or preventing erectile dysfunction, containing LDD175 and a PDE5 inhibitor, as active ingredients.

Other purposes and advantages of the present disclosure will become more obvious with the following detailed description of the invention, claims, and drawings.

### Technical Solution

In accordance with an aspect of the present invention, there is provided a pharmaceutical composition for treating or preventing erectile dysfunction, the composition containing 4-chloro-7-trifluoromethyl-10H-benzo[4,5]furo[3,2-b]indole-1-carboxylic acid (LDD175) or a pharmaceutically acceptable salt thereof as an active ingredient.

As used herein, the term “LDD175” refers to 4-chloro-7-trifluoromethyl-10H-benzo[4,5]furo[3,2-b]indole-1-carboxylic acid represented by chemical formula 1 below, which is a benzofuroindole-based compound. LDD175 is a potent activator of the BK_{Ca} ion pathway, and is known to be involved in the smooth muscle relaxation, which suppresses the contraction of bladder and uterus through activation of BK_{Ca}channels.

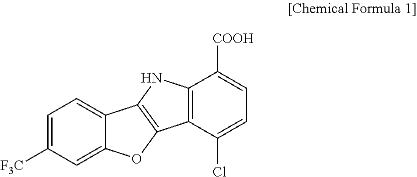 [SMILES: O=C(O)c1ccc(Cl)c2c1[nH]c1c3ccc(C(F)(F)F)cc3oc12]

Herein, the LDD175 activates BK_{Ca} channels of corporal smooth muscle cells, thereby promoting the relaxation of corporal smooth muscles. Herein, the promotion of the relaxation of corporal smooth muscles independently occurs without being affected by the presence or absence of endothelial cells of the corpus cavernosum.

As used herein, the term “pharmaceutically acceptable addition salt” refers to a common acid addition salt or base addition salt, which retains biological efficacy and characteristics of LDD175 and is formed from an appropriate non-toxic organic or inorganic acid, or an appropriate non-toxic organic or inorganic base. Examples of the acid addition salt include acid addition salts derived from inorganic salts, such as hydrochloric acid, hydrobromic acid, hydroiodic acid, sulfuric acid, sulfamic acid, phosphoric acid, and nitric acid; and acid addition salts derived from organic acids, such as p-toluenesulfonic acid, salicylic acid, methanesulfonic acid, oxalic acid, succinic acid, succinic acid, maleic acid, lactic acid, and fumaric acid. Examples of the base addition salt include base addition salts derived from ammonium, potassium, sodium, and quaternary ammonium hydroxide, such as tetramethylammonium hydroxide. The chemical modification of a pharmaceutical compound (that is, a drug) into a salt for the improvement in physical and chemical stability, hygroscopicity, flowability, and solubility of compounds is a well-known technique to pharmaceutical chemists [H. Ansel et. al., Pharmaceutical Dosage Forms and Drug Delivery Systems (6th Ed. 1995) at pp. 196 and 1456-1457], the contents of which are incorporated herein by reference.

As used herein, the term “pharmaceutically acceptable” means that, for example, a pharmaceutically acceptable carrier or an excipient is pharmaceutically acceptable in a patient to which a particular compound is administered, and is substantially non-toxic.

The pharmaceutical composition of the present invention may include a pharmaceutically acceptable carrier in addition to LDD175, which is an active ingredient. The pharmaceutically acceptable carrier is conventionally used at the time of formulation, and examples thereof may include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, and mineral oil.

The pharmaceutical composition of the present invention may further contain a lubricant, a wetting agent, a sweetening agent, a flavoring agent, an emulsifier, a suspending agent, a preservative, and the like, in addition to the above ingredients. Suitable pharmaceutically acceptable carriers and preparations are described in detail in Remington's Pharmaceutical Sciences (19th ed., 1995).

A suitable dose of the pharmaceutical composition of the present invention may vary depending on various factors, such as the method for formulation, manner of administration, the age, body weight, gender, and morbidity of the patient, diet, time of administration, excretion rate, and response sensitivity. Meanwhile, the oral dose of the pharmaceutical composition of the present invention is preferably 0.001 to 1000 mg/kg (body weight) per day.

The pharmaceutical composition of the present invention may be administered orally or parenterally, and examples of the parenteral administration may include intravenous, subcutaneous, intramuscular, intraperitoneal, and transdermal injections.

The concentration of LDD175, which is an active ingredient contained in the composition of the present invention, may be determined considering the therapeutic purpose, the condition of the patient, the required period, or the like, and is not limited to a specific range of concentration.

The pharmaceutical composition of the present invention is formulated into a unit dosage form or in a multidose container, using a pharmaceutically acceptable carrier and/or excipient according to the method that can be easily conducted by person having ordinary skills in the art to which the present invention pertains. Here, the dosage form may be a solution in an oily or aqueous medium, a suspension, an emulsion, an extract, a powder, a granule, a tablet, or a capsule, and may further include a dispersant or a stabilizer.

In accordance with another aspect of the present invention, there is provided a co-administration preparation for treating or preventing erectile dysfunction, containing: (i) 4-chloro-7-trifluoromethyl-1 OH-benzo[4,5]furo[3,2-b]indole-1-carboxylic acid; and (ii) a phosphodiesterase type 5 (PDE5) inhibitor, as active ingredients.

As used herein, the term “PDE5 inhibitor” refers to a drug which functions to treat erectile dysfunction by blocking the cyclic GMP degradation activity of cGMP-specific phosphodiesterase type 5 (PDE5) in smooth muscle cells of corpus cavernosal blood vessels. Specific examples of the PDE5 inhibitor include avanafil, lodenafil, mirodenafil, sildenafil, tadalafil, vardenafil, udenafil, or zaprinast, but are not limited thereto.

Herein, the co-administration of LDD175 and the PDE inhibitor synergistically induces the corporal smooth muscle relaxation effect.

In accordance with still another aspect of the present invention, there is provided a method for treating erectile dysfunction, the method including administering a pharmaceutical composition to an erectile dysfunction patient, the pharmaceutical composition containing 4-chloro-7-trifluoromethyl-10H-benzo[4,5]furo[3,2-b]indole-1-carboxylic acid or a pharmaceutically acceptable salt.

In accordance with still another aspect of the present invention, there is provided a method for treating erectile dysfunction, the method including administering a co-administration preparation to an erectile dysfunction patient, the preparation containing (i) 4-chloro-7-trifluoromethyl-1 OH-benzo[4,5]furo[3,2-b]indole-1-carboxylic acid and (ii) a phosphodiesterase type 5 (PDE) inhibitor.

In accordance with still another aspect of the present invention, there is provided a use of 4-chloro-7-trifluoromethyl-10H-benzo[4,5]furo[3,2-b]indole-1-carboxylic acid for preparing an erectile dysfunction drug.

In accordance with sill another aspect of the present invention, there is provided a use of (i) 4-chloro-7-trifluoromethyl-10H-benzo[4,5]furo[3,2-b]indole-1-carboxylic acid; and (ii) a phosphodiesterase type 5 (PDE5) inhibitor, for preparing an erectile dysfunction inhibitor.

### Advantageous Effects

Features and advantages of the present invention are summarized as follows.

(i) The present invention is directed to a pharmaceutical composition for treating or preventing erectile dysfunction, containing LDD175 as an active ingredient. Furthermore, the present invention is directed to a co-administration preparation for treating or preventing erectile dysfunction, containing LDD175 and a PDE5 inhibitor, as active ingredients.

(ii) The LDD175 of the present invention had an excellent corporal smooth muscle relaxation effect, and thus significantly improved an erectile function in the animal model.

(iii) Since the LDD175 of the present invention acts on BK_{Ca} channels, which are less expressed in cardiovascular tissues, the LDD175 is expected to have little adverse cardiovascular effects.

(iv) The corporal smooth muscle relaxation effect of LDD175 of the present invention was not affected by the presence or absence of endothelial cells of the corpus cavernosum.

(v) The LDD175 of the present invention exhibited an erectile dysfunction therapeutic ability equivalent to that of udenafil, which is a PDE5 inhibitor, and thus showed a synergistic corporal smooth muscle relaxation effect when being co-administered with udenafil.

### BRIEF DESCRIPTION OF THE DRAWINGS

FIG. 1a shows a concentration-response curve for LDD175 and the rabbit corporal smooth muscle (CSM) strip (n=10, 10⁻⁷ M=3.7±1.4%, 10⁻⁶ M=14.5±2.6%, 10⁻⁵ M=26.3±2.0%, and 10⁻⁴ M=54.0±3.0%). The relaxation effect is expressed as the % of the phenylephrine (PE)-induced contraction.

FIG. 1b illustrates results by measuring effects of endothelium-denudation (n=10) or IbTX (n=7) on the relaxation response induced by LDD175. Removal of the endothelium did not significantly affect the relaxation potencies (DMSO only, n=5, 15.7±2.9%; intact endothelium, n=7, 45.9±6.2%, P<0.05 vs. DMSO only; denuded endothelium, n=10, 41.0±4.9%, P>0.05 vs. intact endothelium).

FIG. 1c illustrates that the relaxation response induced by LDD175 was significantly inhibited after the pretreatment of the strips with IbTX, a selective blocker of BK_{Ca} channels (LDD175, n=7, 45.9±6.2%; LDD175+IbTX, n=7, 23.6±3.3%, P<0.05 vs. LDD175). The measurement values were expressed as mean±SE. CSM: corporal smooth muscle, IbTX: iberiotoxin.

FIGS. 2a and 2b illustrate results of relaxation effects induced by LDD175 (n=10, 10⁻⁴ M, 54.0±3.1%) and udenafil (n=6, 10⁻⁶ M, 34.5±3.9%; 10⁻⁶ M, 67.1±1.7%) in phenylephrine-contracted rabbit CSM strips.

FIG. 2a shows that 10⁻⁴ M LDD175 had a greater relaxation effect than 10⁻⁶ M undenafil (P<0.01).

FIG. 2b shows that the combination of 10⁻⁵ M LDD175 and 10⁻⁶ M undenafil had synergistic effects on relaxation (n=7, 10⁻⁵ M LDD, 32.7±3.8%; 10⁻⁶ M undenafil, 21.6±2.7%; 10⁻⁵ M LDD+10⁻⁶ M undenafil, 50.4±4.5%, P<0.05). These results are expressed as the % relaxation of the phenylephrine-induced contraction.

FIG. 3a illustrates representative current trace results recorded at a holding potential of −60 mV with the external solution containing 5 mM K.

FIG. 3b shows a current-voltage (I-V) curve measured by a 500 ms ramp pulse of −100 mV to +80 mV. LDD175 activated outward-rectifying K⁺ currents by a maxim of 952.1% at +60 mV (n=13, P<0.05 vs. control).

FIG. 3c shows that LDD175-activated current was completely blocked by TEA (1 mM, n=6, 78.2%, P<0.05) or IbTX (100 nM, n=4, 78.1%, P<0.05).

FIG. 3d shows that LDD175 activated whole-cell K⁺ currents in a dose-dependent manner in CSM cells.

FIG. 3e is a graph showing a summary of peak current densities at +60 mV (n=10, control, 24.5±5.8 pA/pF; 0.1 μM, 30.9±6.5 pA/pF; 1 μM, 45.1±8.2 pA/pF; 10 μM, 178.5±30.0 pA/pF; 30 μM, 257.3±48.3 pA/pF; 100 μM, 265.5±53.4 pA/pF, at 60 mV, P*<0.05 vs. control). The measurement values were expressed as mean±S.E.M. CSM: corporal smooth muscle, IbTX: iberiotoxin, TEA: tetraethylammonium: w/o: wash out.

FIG. 4a is a graph showing a typical current-voltage curve obtained by using a 500 ms ramp pulse of −80 mV to +80 mV. Membrane currents were recorded in the absence and presence of 30 μM NS1619 at a holding potential of +60 mV (n=7, p<0.05 vs. control).

FIG. 4b shows that the NS1619-activated current was completely blocked by 100 nM IbTX.

FIGS. 4c and 4d are graphs showing summaries of peak current densities at a holding voltage of +60 mV (panel C) and −40 mV (panel D). The efficacy and efficiency of LDD175 were significantly higher than those of NS1619 (−40 mV, NS1619: n=7, 1.5±0.6 pA/pF, 1.3-fold vs. control, P=0.6; LDD175: n=13, 23.9±5.8 pA/pF, 42.8-fold vs. control, P<0.05). The measurement values were expressed as mean±S.E.M. P*<0.05 vs. con: control. IbTX: iberiotoxin, w/o: wash out.

FIGS. 5a and 5b show in vivo animal model experiment results of cavernous nerve stimulation for evaluating erectile function after pretreatment with LDD175 and undenafil under sub-maximal EFS (2 Hz, 1 V, and 60 seconds) and maximal EFS (10 Hz, 6 V, and 60 seconds). FIG. 5a: maximal ICP (n=8, 57.8±6.6 vs. 48.9±5.4, P<0.05). FIG. 5b: maximal ICP/ABP ratio (n=8, 45.3±5.8 vs. 41.0±5.0, P<0.05). FIG. 5c: AUC of the ICP/ABP ratio (n=8, 1444.4±133.3 vs. 1093.7±123.1, P<0.05). P*<0.05 vs. con: control. ABP: artificial blood pressure, AUC: area under curve, EFS: electrical field stimulation, ICP: intracavernosal pressure.

FIG. 6 illustrates measurement results of intracavernosal pressures depending on electrical stimulation conditions in normal white rats.

FIGS. 7a to 7e illustrate measurement results of intracavernosal pressures of white rats with diabetes in the same manner as in FIG. 6.

FIG. 7a: DM control,

FIG. 7b: 1 mg/kg sidenafil,

FIG. 7c: 5 mg/kg sildenafil,

FIG. 7d: 5 mg/kg LDD175,

FIG. 7e: 10 mg/kg LDD175.

FIG. 8 illustrates measurement results of intracavernosal pressures after the injection of 30 mg/kg tetraethylammonium (TEA), which is a BK_{Ca} channel inhibitor, and after 10 minutes, the injection of LDD175 (10 mg/kg).

FIG. 9 illustrates measurement results of intracavernosal pressures after co-administration of LDD175 (5 mg/kg) and sildenafil (1 mg/kg).

FIG. 10 illustrates graphs showing the summary of the results of FIGS. 7 to 9.

### DETAILED DESCRIPTION

Hereinafter, the present invention will be described in detail with reference to examples. These examples are only for illustrating the present invention more specifically, and it will be apparent to those skilled in the art that the scope of the present invention is not limited by these examples.

### Examples

### Methods

1. Isolation of Corpus Cavernosal Strip from Animal Model

Sexually mature male New Zealand white rabbits (3.0±0.3 kg) were killed with an air embolism in the ear vein. The entire penis was then surgically excised, and then cleaned by removing the corpus cavernosum and urethra. Then, the corporal tissue was carefully dissected from the surrounding tunica. Four corpus cavernosal strips with approximately equal sizes (2×2×10 mm) were obtained from each penis.

2. Contraction Studies Using Organ Bath

Rabbit corpus cavernosal strips were separately prepared for organ-bath studies. Each corporal strip was mounted in a 10 ml-organ chamber filled with Krebs buffer (composition in mmol/l: 118.4 NaCl, 4.7 KCl, 2.5 CaCl₂, 1.2 KH₂PO₄, 1.2 MgSO₄, 24.9 NaHCO₃, and 11.1 D-glucose), with one end fixed to a tissue holder and the other secured to a force transducer. The force transducer was connected to an appropriately calibrated four-channel polygraph (Power-Lab; AD Instruments, Sydney, NSW, Australia), in which the transducer output was recorded. The corpus cavernosal strips were kept in the organ bath with Krebs solution maintained at 36.5° C. by a thermo-regulated water circuit and by continuously bubbling with a mixture of 95% O₂ and 5% CO₂. Each corporal strip was stretched to an optimal isometric tension of 1.0 g and was equilibrated for 60 min. During the equilibration, the tissues were washed with fresh Krebs solution every 15 min, and the tension was adjusted if necessary.

The vasodilatory effect of LDD175 was studied by cumulative addition of the sample at concentrations ranging from 10⁻⁷ to 10⁻⁴ M to endothelium-intact strips and endothelium-denuded strips after pre-contraction with 10⁻⁵ M phenylephrine (PE). The endothelial lining of the corpus cavernosum was removed by rubbing the strip between the thumb and index finger for 20 seconds and soaking the same in 0.3% solution of 3[(3-cholamidopropyl)-dimethylammonio]-l-propanesulphonate (CHAPS) for 10 seconds. It was confirmed whether the endothelium was functionally completely removed for all strips by pre-contracting the strips with 10⁻⁵ M SD PE and then adding acetylcholine (10⁻⁶ M) thereto. If the tissue did not respond to acetylcholine, it was accepted as endothelium-free and was subsequently used in the study. In order to demonstrate the role of K⁺ channels affecting LDD175-induced relaxation, the strips were pre-treated with 100 nM iberiotoxin (IbTX), a BK channel-specific inhibitor, for 30 minutes, before PE was added. After the addition of IbTX, LDD175 was added to strips pre-contracted with PE. The relaxation effect of the PDE5 inhibitor (udenafil, 10⁻⁶ and 10⁻⁵ M) was also separately measured in the same manner, and compared to that of LDD175. The synergistic relaxation effect between 10⁻⁵ M LDD175 and 10⁻⁶ M udenafil was also investigated.

3. Preparation of Corpus Cavernosal Strip from Human Tissue, and Cell Culture

Human corporal tissues were obtained from the corpus cavernosa of patients with organic erectile dysfunction undergoing implantation of penile prostheses. Homogeneous explant cell cultures of human corporal smooth muscle (CSM) cells were prepared as follows (Palmer et al., 1994; Sung et al., 2011). Radial sections of approximately 3×3×10 mm were excised from the mid-penile shafts of the respective resected sample. These samples were tissues including smooth muscles, endothelium, and connective tissues, with occasional nerve fibers. The tissues were washed, cut into 1-2 mm pieces, and placed in tissue culture dishes filled with Dulbecco's modified Eagle's medium (DMEM, Gibco, Carlsbad, Calif., USA) supplemented with 10% fetal bovine serum and antibiotics (100 units/mL penicillin, streptomycin; Gibco), under 5% O₂ and 5% CO₂. After the explants were attached to the substrate (usually 1-2 days), additional medium was added. The smooth muscle cells were observed migrating from the explants, and underwent division. The cells were subsequently detached via a trypsin/EDTA protocol to establish secondary cultures from the explants. These cultures were morphologically homogeneous. The cobblestone morphology, which is the characteristic of endothelial cells, and the flattened and spread-out shape, which is the characteristic of fibroblasts, were not observed. Cellular homogeneity was further verified by observing smooth muscle-specific α-actin and myosin immuno-responsiveness. While the cultures were maintained for four or more passages, all measured pharmacological and molecular properties were evaluated in the intact tissues that were maintained in the cultures, for example, cyclic adenosine monophosphate formation (Palmer et al., 1994), calcium mobilization (Zhao and Christ, 1995), expression or function of the gap junction protein, connexin (Brink et al., 1996), and K+ channel activity (Lee et al., 1999).

4. Electrophysiological Studies

The Ca²⁺-activated potassium channel (BK_{Ca}) was measured by using the conventional whole-cell patch-clamp technique (Sung et al., 2011). Patch electrodes were made from borosilicate glass capillary tubing (World Precision Instruments, Sarasota, Fla., USA), and had 2.5-5 MO resistance. The cell suspension was placed into a small chamber (0.6 ml) on the stage of an inverted microscope (TMD Diaphot, Nikon, Tokyo, Japan). The membrane currents in smooth muscle cells were recorded using a patch clamp amplifier (Axopatch-1 D, Axon Instruments, Foster City, Calif., USA). The liquid junction potential between the pipette solution and the bath solution was only about 3 mV, and thus uncorrected. In each experiment, whole-cell configuration was not achieved until the seal resistance became larger than 5 GO. The pCLAMP software (Axon Instruments) and Digidata-1322A (Axon Instruments) were used for data acquisition and application of command pulses. Membrane currents were measured during ramping, and filtered at 5 kHz (−3 dB frequency). The current signals were filtered at 5 kHz, digitized, and analyzed on a personal computer using pCLAMP and Origin software (Microcal Software, Northampton, Mass., USA). The standard bath solution was prepared to contain 135 mmol NaCl, 5 mmol KCl, 10 mmol HEPES, 1.8 mmol CaCl₂, 1 mmol MgCl₂, and 5 mmol glucose, per liter, at pH 7.4 adjusted with NaOH. The pipette (internal) solution was prepared to contain, per liter, 140 mmol KCl, 10 mmol HEPES, 5 mmol K₂ATP, 2 mmol MgCl₂, 5 mmol ethyleneglycol-bis-[2-aminoethyl ether]-N,N′-tetraacetic acid (EGTA), and 0.5 mmol GTP, at pH 7.2 adjusted with KOH. The Ca²⁺-free concentration in the pipette solution was adjusted to 3.15 nM by adding an appropriate amount of CaCl₂, determined using the Max Chelator Sliders software (C. Patton, Stanford University).

5. In Vivo Animal Experiments Using Cavernous Nerve Stimulation Model

The erectile response was measured using Sprague-Dawley rats. The detailed methodology of measuring the erectile response has been described previously (Rehman et al., 1997). The 8-10 wk aged rats were anesthetized with intramuscular injection of Zoletil 100 mg/kg (Virbac, Carros, France), and placed on a thermally regulated surgical table. Systemic arterial blood pressure (ABP) was monitored via carotid artery cannulation (polyethylene-50 tubing). The prostate and bladder were exposed through a midline abdominal incision. The major pelvic ganglion, pelvic nerves, and cavernous nerve were identified as posterolateral to the prostate on each side. An electric stimulator with stainless steel bipolar hook electrodes was placed around the cavernous nerve for electrical stimulation. To monitor intracavernosal pressure (ICP), a 25-gauge cannula was filled with 250 U/ml, connected to polyethylene-50 tubing, and inserted into one side of the crura. Systemic arterial blood pressure (ABP) and intracavernosal pressure (ICP) were measured with a transducer connected to a PowerLab® (AD Instruments, Colorado Spring, Colo., USA), which is a computerized system for data acquisition. Real-time display and documentation of pressure were conducted with Chart™ 6 software. Sub-maximal electrical field stimulation (EFS) was used with 1 V, 2 Hz, 5 ms pulse width, and duration of 60 seconds. Sub-maximal electrical field stimulation was applied 10 min after a series of control (buffer solution alone), LDD175 (10 mg/kg), and udenafil (1 mg/kg) injections. At the end of sub-maximal stimulation, the full response was measured using the EFS of 6 V, 10 Hz, 5 ms pulse width, with duration of 60 seconds. Buffer solution, LDD175, and udenafil were administered at intervals of 30 min. The ratio of maximal ICP to mean arterial BP at the peak of the erectile response was calculated in order to control for variations in ABP.

6. Diabetic Erectile Dysfunction Rat Model

The 8-wk aged male Sprague-Dawley rats were administered with an intraperitoneal injection of streptozotocin (65 mg/kg body weight) dissolved in 5 mM citric acid buffer (pH 4.5), thereby inducing insulin dependent diabetes mellitus (IDDM). Rats of the normal control were injected with only 5 mM citric acid buffer (pH 4.5) by the same method. On day 2 of breeding, the rats were fasted for 16 hours, and the blood was collected from the tail vein to conduct the blood glucose test. Only rats with a blood glucose level of 250 mg/de or higher were selected for a diabetes induced group.

7. Drugs and Solutions

LDD175 was provided from by AnyGen Co., Ltd., (Gwangju, Korea), and udenafil from Dong-A Pharmaceutical (Seoul, Korea). All other chemical agents were purchased from Sigma Chemical (St. Louis, Mo., USA). For the in vitro organ bath and electrophysiological studies, LDD175 was dissolved in dimethyl sulfoxide (DMSO) as described in the previous prior art documents (Ahn et al., 2011). NS1619 was dissolved in ethanol. The stock solution was diluted immediately before use. The final concentration of the solvent was less than 0.1%, and this bath concentration of solvent did not affect smooth muscle tone or ion-channel activity. For the in vivo functional studies, LDD175 was dissolved in 10% Tween 20, and all other drugs were dissolved in distilled water.

8. Statistical Analysis

All data were expressed as means±standard error (SE), and were analyzed using IBM Statistical Package for the Social Sciences (SPSS) Statistics version 21.0 (IBM, Armonk, N.Y., USA). The two-tailed Student t-test and paired t-test were used as appropriate to compare group means with differences considered significant at p<0.05.

### Results

1. Relaxation Effects of LDD175 on Rabbit CSM Through Bk_{Ca} Channel

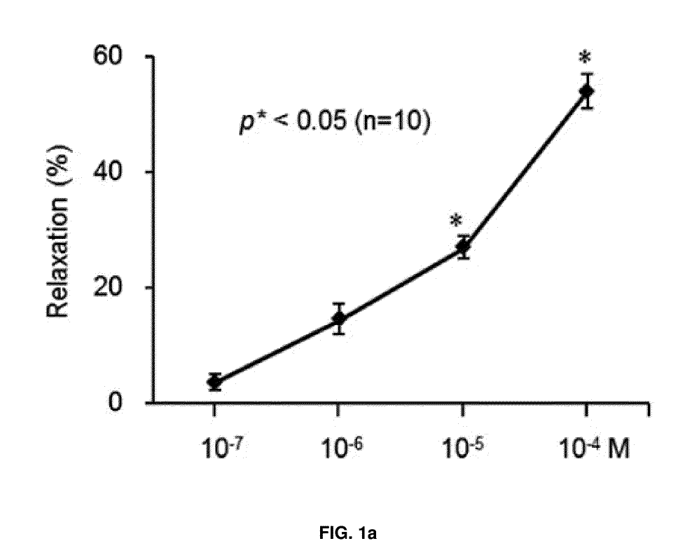

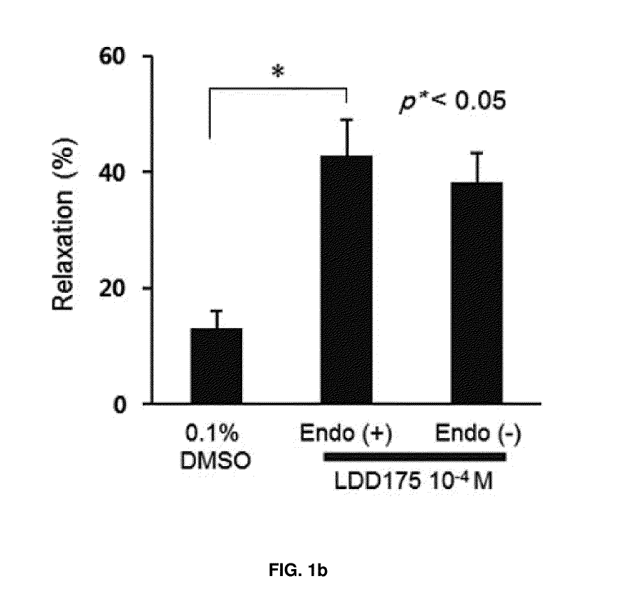

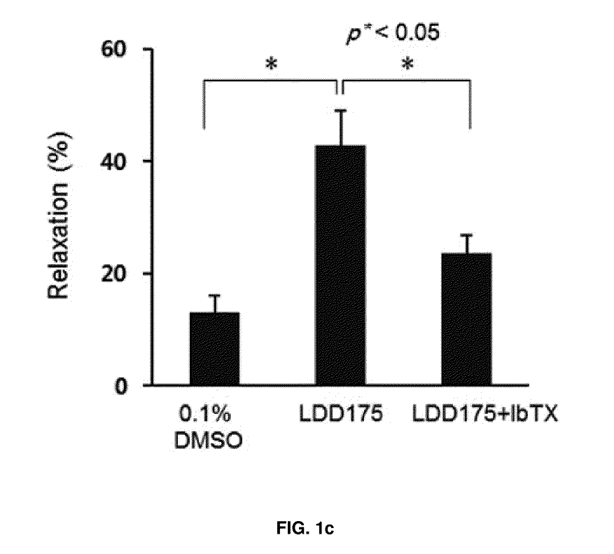

In the organ bath studies, pre-contracted rabbit CSM strips were relaxed by LDD175 in a dose-dependent manner from 10⁻⁷ M to 10⁻⁴ M (n=10, 10⁻⁷ M=3.7±1.4%, 10⁻⁶ M=14.5±2.6%, 10⁻⁵ M=26.3±2.0%, and 10⁻⁴ M=54.0±3.0%, FIG. 1(a)); and 10⁻⁴ M LDD175 induced endothelium-independent relaxation of rabbit corporal smooth muscle strips (DMSO only, n=5, 15.7±2.9%; intact endothelium, n=7, 45.9±6.2%, p<0.05 vs. DMSO only; denuded endothelium, n=10, 41.0±4.9%, p>0.05 vs. intact endothelium, FIG. 1(b)). However, the pretreatment of IbTX, a selective blocker of BK_{Ca} channels, significantly inhibited the relaxation response of 10⁻⁴ M LDD175 (LDD175, n=7, 45.9±6.2%; LDD175+IbTX, n=7, 23.6±3.3%, p<0.05 vs. LDD175, FIG. 1c).

2. Comparison with PDE5 Inhibitor and Synergistic Effect with PDE5 Inhibitor

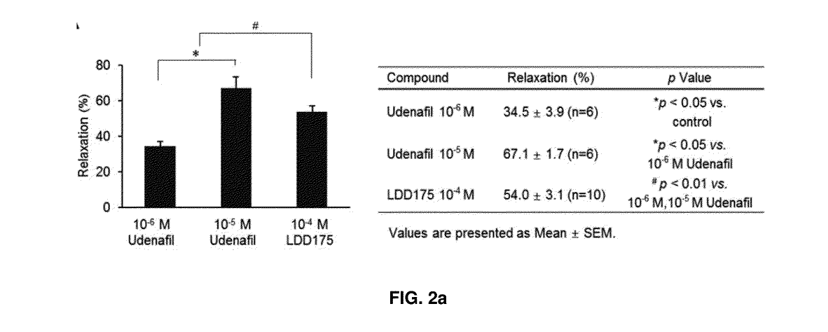

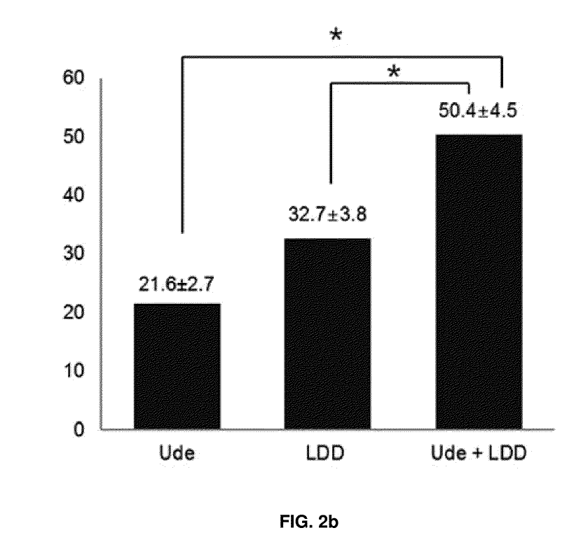

Udenafil also induced the relaxation of rabbit CSM strips in a dose-dependent manner (n=6, 10⁻⁶ M, 34.5±3.9%; 10⁻⁵ M, 67.1±1.7%). The treatment with 10⁻⁴ M LDD175 (n=10, 54.0±3.1%) had a greater relaxation effect than the treatment with 10⁻⁶ M udenafil (p<0.01, FIG. 2a). The co-treatment of 10⁻⁵ M LDD175 and 10⁻⁶ M udenafil had a synergistic relaxation effect (n=7, 10⁻⁵ MLDD, 32.7±3.8%; 10⁻⁶ M udenafil, 21.6±2.7%; 10⁻⁵ M LDD+10⁻⁶ M udenafil, 50.4±4.5%, p<0.05, FIG. 2b).

3. Effect of LDD175 on BK_{Ca} Currents in Human CSM Cells

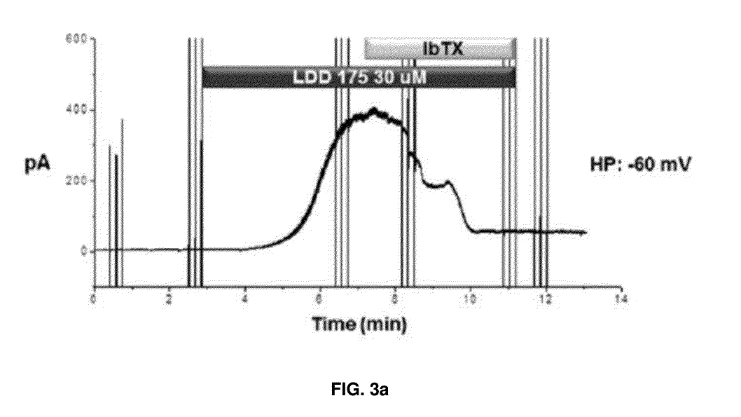

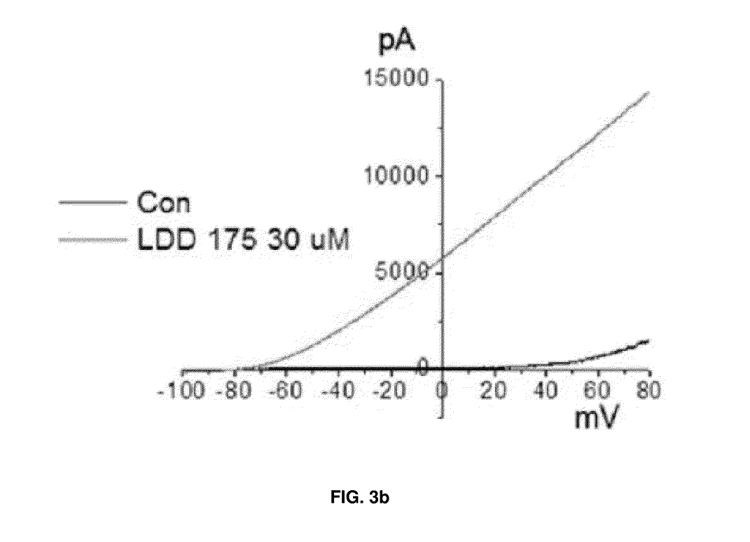

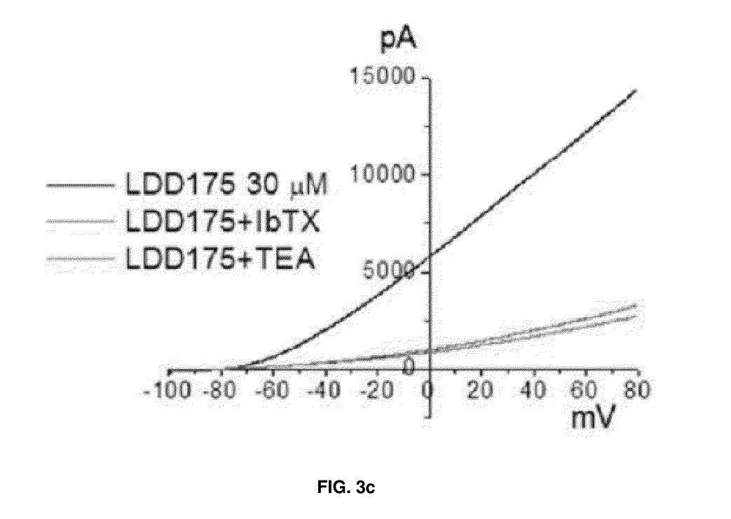

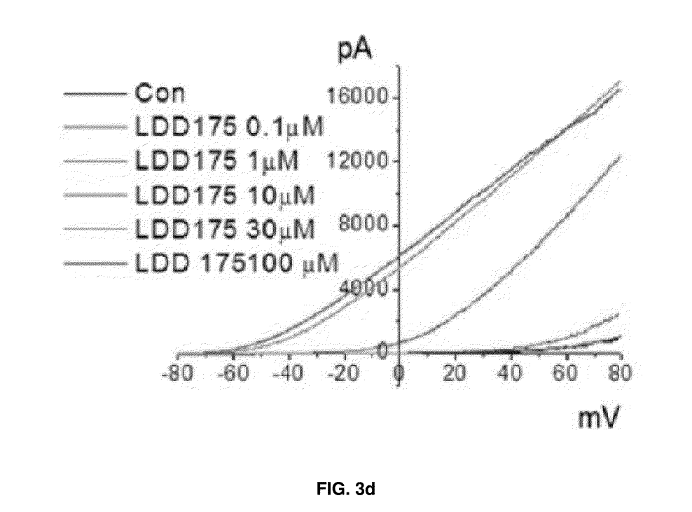

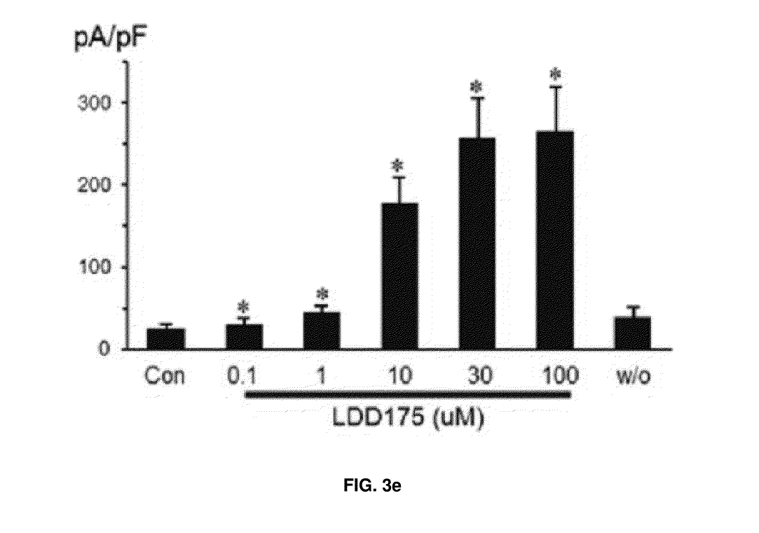

To directly investigate the effect of LDD175 on the activity of the BK_{Ca} channel, whole-cell patch-clamp recordings were conducted in cultured human CSM cells. At a holding potential of −60 mV with the external solution containing 5 mM K⁺, extracellular application of 30 μM LDD175 significantly increased outward currents at all membranes, and this effect was completely inhibited by the treatment with 100 nM IbTX (FIG. 3a). As shown in FIG. 3b, the current-voltage (I-V) relationship showed that LDD175 activated outward rectifying K+ currents, by a maximum of 952.1% at +60 mV (n=13, p<0.05 vs. control). 1 mM tetraethylammonium (TEA) or 100 nM IbTX suppressed these currents by 78.2% (n=6, p<0.05) and 78.1% (n=4, p<0.05), respectively (FIG. 3c). The effect of LDD175 on the outward current was dose-dependent, and the increased currents were readily reversed by drug washout (FIG. 3d). FIG. 3e is a graph showing a summary of average peak current densities, which were gradually increased as a higher concentration of LDD175 was added (n=10, control, 24.5±5.8 pA/pF; 0.1 μM, 30.9±6.5 pA/pF; 1 μM, 45.1±8.2 pA/pF; 10 μM, 178.5±30.0 pA/pF; 30 μM, 257.3±48.3 pA/pF; 100 μM, 265.5±53.4 pA/pF, at 60 mV, P*<0.05 vs. control).

4. Comparison of Effects of NS1619 and LDD175 on BK_{Ca} Channels in Human CSM Cells

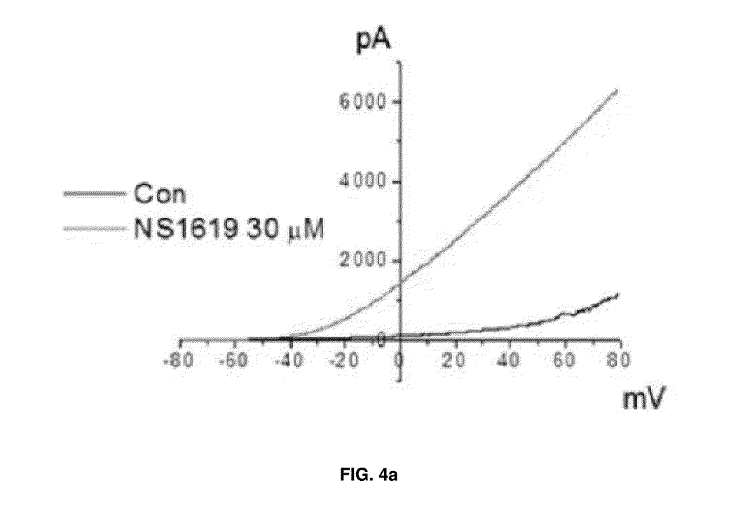

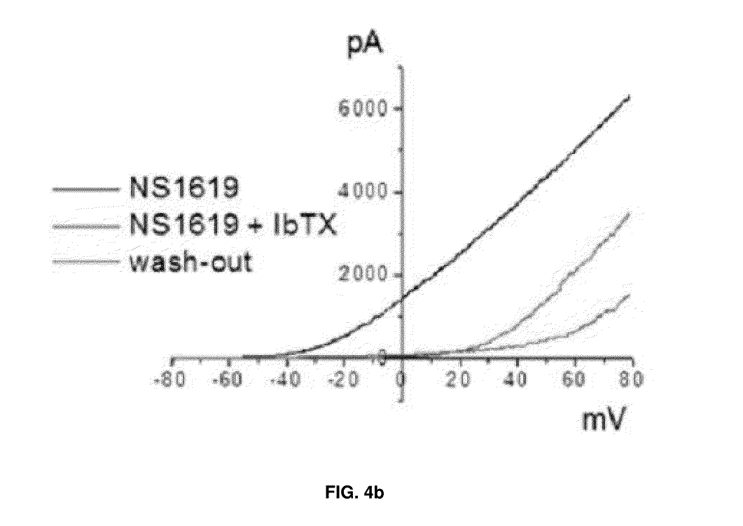

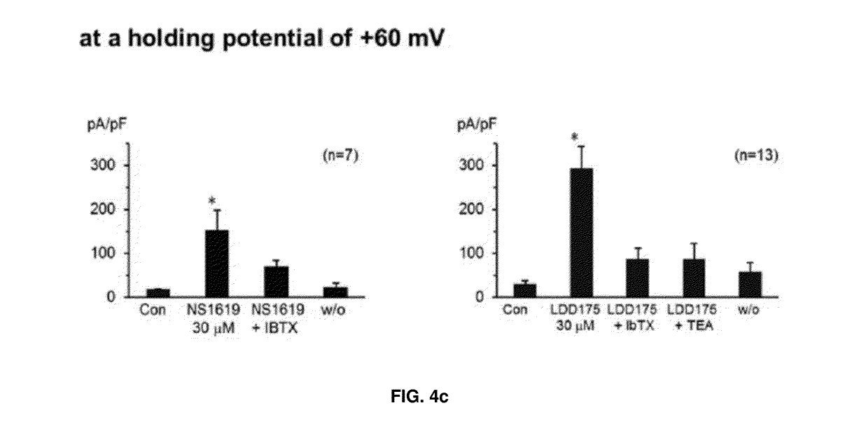

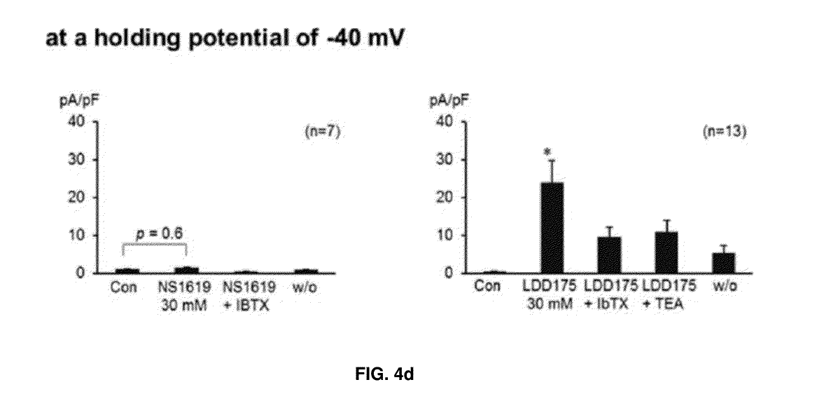

To compare the effect of LDD175 with other BK_{Ca} channel activators, the electrophysiological effect of NS1619, a typical selective BK_{Ca} channel opener, was tested. Extracellular application of 30 μM NS1619 also markedly increased the whole-cell currents at a holding potential of +60 mV by 880.0%, similar to activation by LDD175 (n=7, p<0.05 vs. control, FIG. 4a). This increase of outward currents by NS1619 was completely inhibited by the addition of 100 nM IbTX (FIG. 4b). At −40 mV, which is near the resting membrane potential (or more physiological potentials) of the cell, 30 μM LDD175 still activated currents (n=13, 23.9±5.8 pA/pF, p<0.05), but 30 μM NS1619 did not change the currents (n=7, 1.5±0.6 pA/pF, p=0.6, FIG. 4d). These results demonstrated that LDD175 might be activated under physiological conditions, unlike NS1619.

5. In Vivo Nerve Stimulation Rat Model

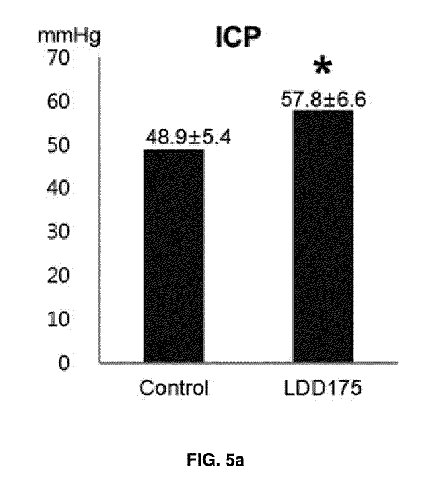

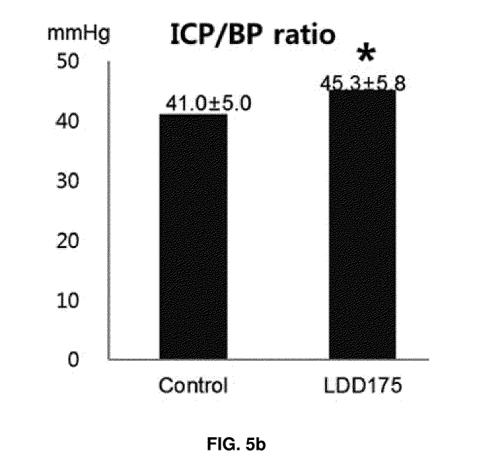

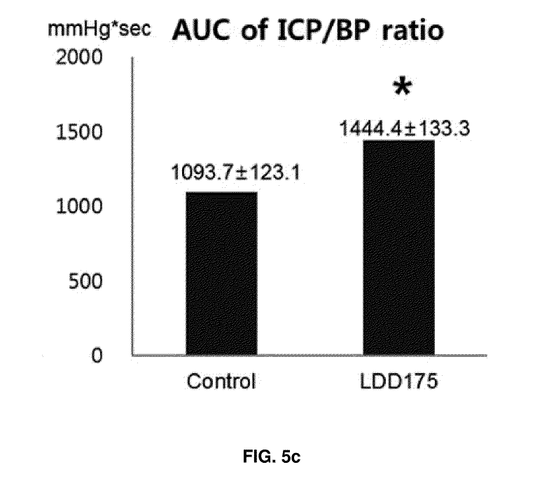

With sub-maximal electrical field stimulation, an intravenous administration of LDD175 improved the erectile function of rats in terms of maximal ICP (n=8, 57.8±6.6 mmHg vs. 48.9±5.4 mmHg, p<0.05, FIG. 5a), maximal ICP/ABP ratio (n=8, 45.3±5.8% vs. 41.0±5.0%, p<0.05, FIG. 5b), and area under curve (AUC) of ICP/ABP ratio (n=8, 1444.4±133.3 vs. 1093.7±123.1, p<0.05, FIG. 5c). There was no difference between the effect of 10 mg/kg LDD175 and 1 mg/kg udenafil regarding all aspects of interest, including maximal ICP, maximal ICP/ABP ratio, and the AUC of ICP/ABP ratio.

6. Effect of LDD175 on Erectile Function in Rat Model with Diabetic Erectile Dysfunction

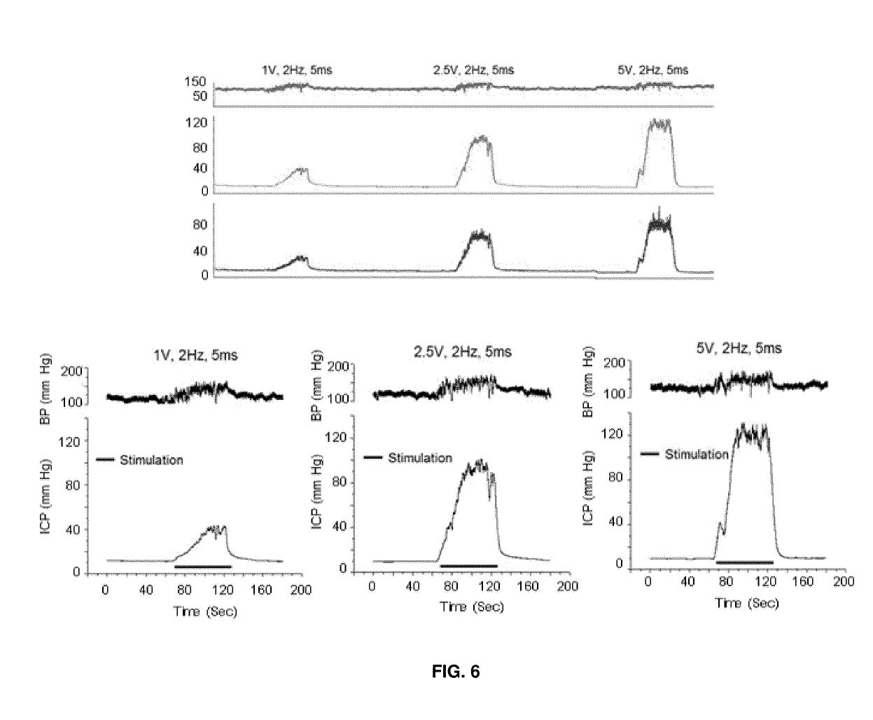

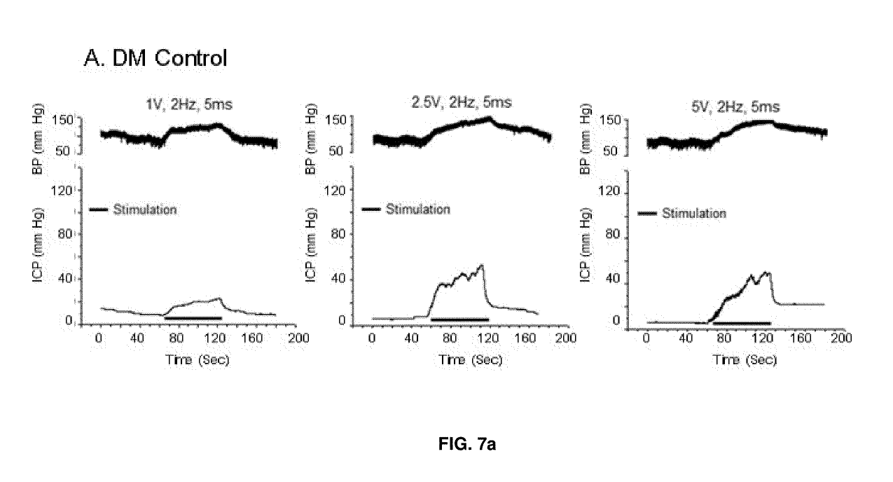

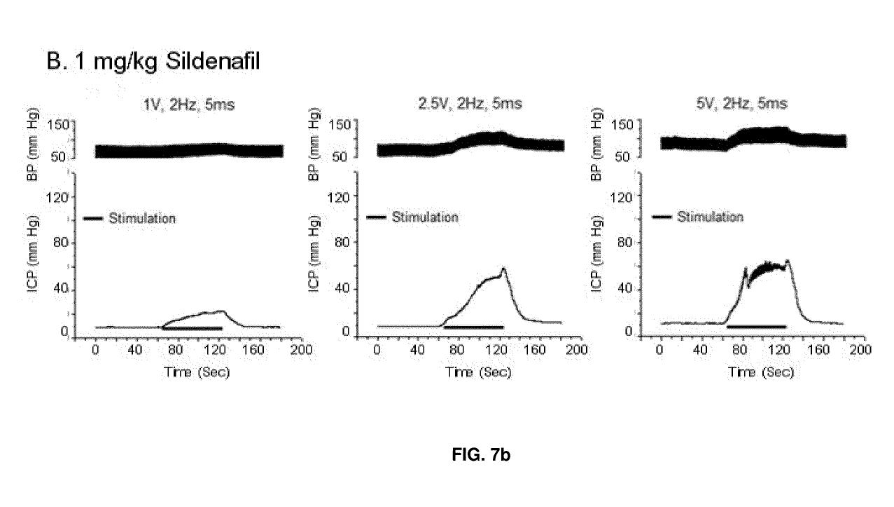

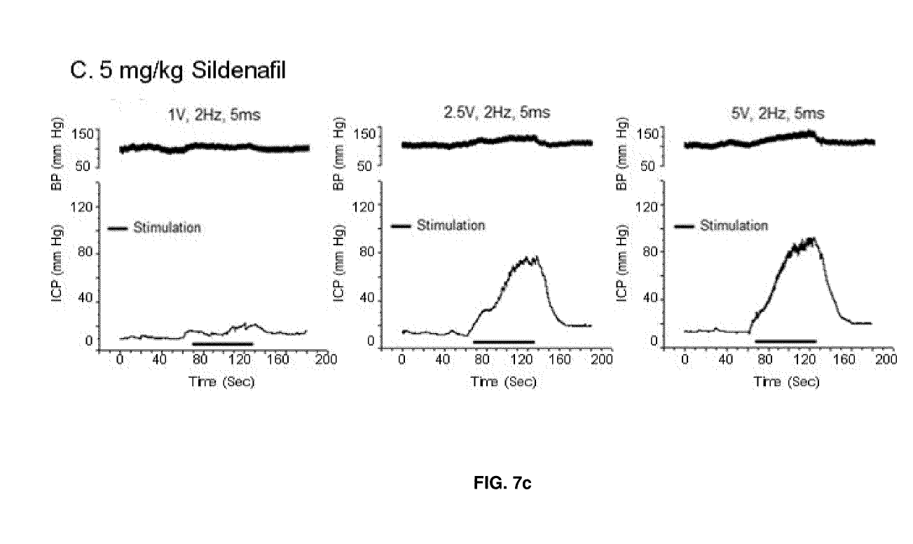

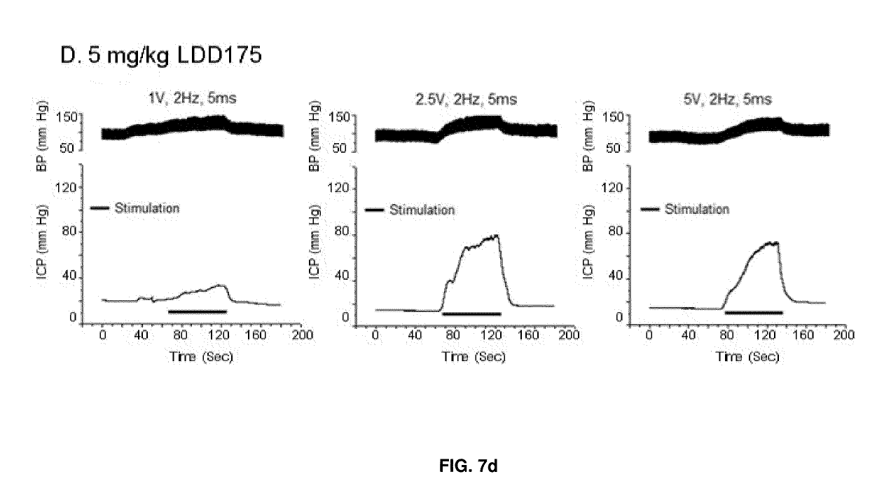

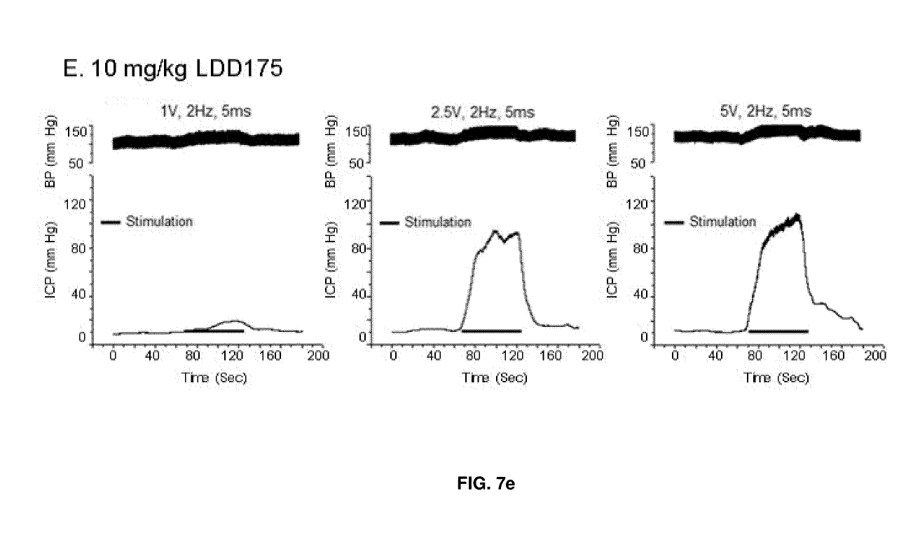

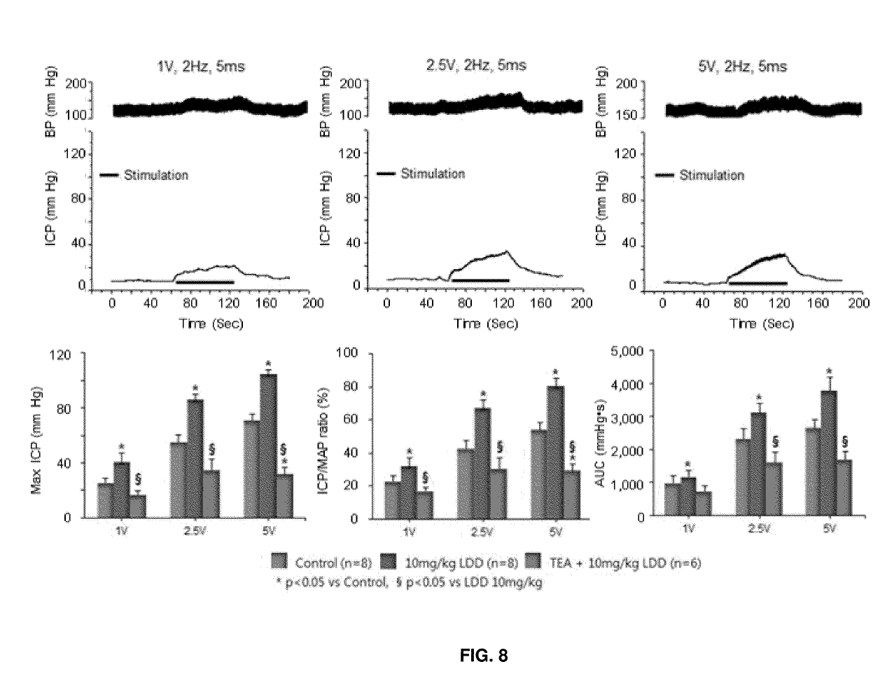

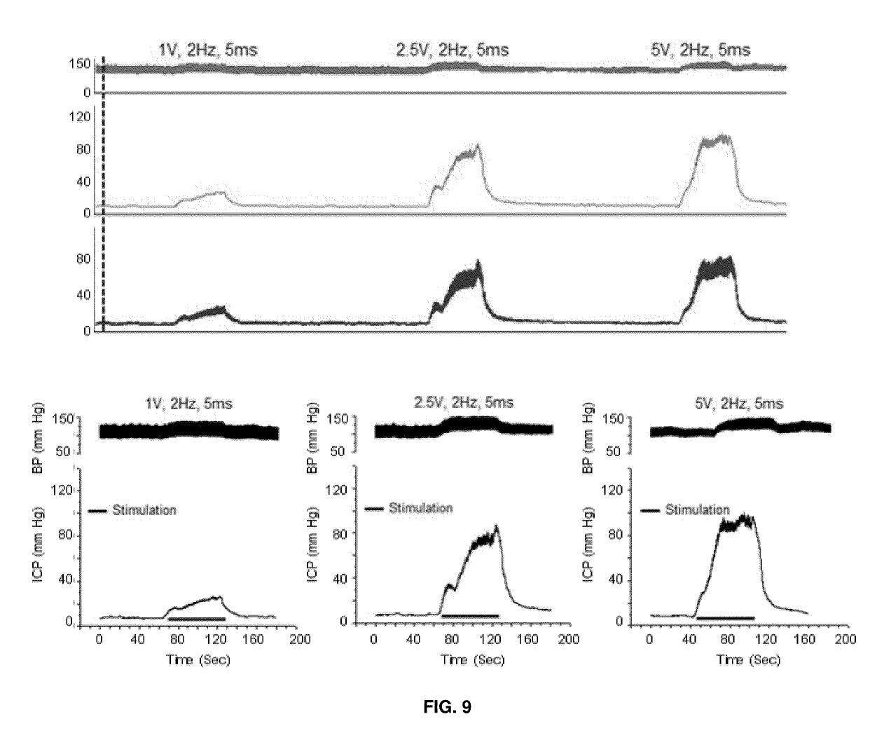

FIG. 6 illustrates intracavernosal pressures measured depending on electrical stimulation conditions in normal white rats. As a result of measuring the intracavernosal pressure of the white rats with diabetes by the same method, the erectile function was significantly reduced compared with the normal control. The maximal ICP/ABP ratios was measured to be 1 V: 18.0±3.3 mmHg, 2.5 V: 34.9±4.2 mmHg, and 5 V: 45.7±4.5 (n=8) (FIG. 7a). The injection of sildenafil, a PDE5 inhibitor, significantly increased the intracavernosal pressure. The maximal intracavernosal pressure, the maximal ICP/ABP, and the AUC, somewhat increased in 1 mg/kg injection group rather than in the control, but no statistical significance was shown (maximal ICP/ABP ratio; 1 V: 23.8±2.6 mmHg, 2.5 V: 46.7±7.3 mmHg, 5 V: 55.4±5.2, n=6, p>0.05), and the erectile function of the 5 mg/kg injection group was improved to the erectile function level of the normal group (1 V: 32.0±6.1 mmHg, 2.5 V: 64.0±5.5 mmHg, 5 V: 69.9±5.4, n=7, p<0.05) (FIGS. 7b and 7c). The group injected with LDD175 by the same method also had a significantly increased intracavernosal pressure, and the 10 mg/kg injection group was more effective than the 5 mg/kg injection group (FIGS. 7d and 7e). In order to verify the action mechanism of in vivo efficacy of LDD175, tetraethylammonium (TEA, 30 mg/kg), which is a BK_{Ca} channel inhibitor, was injected, and after 10 minutes, LDD175 (10 mg/kg) was again injected. As a result, the effect of LDD175 was completely inhibited (FIG. 8).

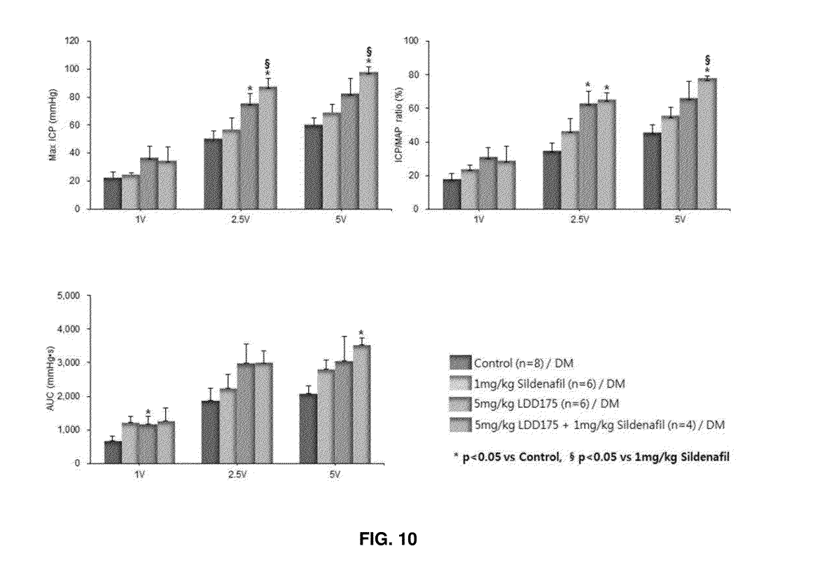

The co-administration of low concentrations of LDD175 (5 mg/kg) and sildenafil (1 mg/kg) had a significantly increased intracavernosal pressure compared with LDD175 or sildenafil alone. FIG. 10 illustrates the summary of the foregoing study results, verifying that, like the study results in normal rats, the erectile function of the rat model with diabetic erectile dysfunction was recovered to the level of the normal group. The present study results suggest LDD175 can be a novel candidate as a therapeutic agent for patients who do not respond to PDE5 inhibitors, which are erectile dysfunction drugs, as well as patients with diabetic erectile dysfunction.

### Discussion

The main results of the current study was that LDD175 relaxed CSM strips that had been pre-contracted by PE in organ bath studies, and this relaxation was completely blocked by IbTX, a BK_{Ca} channel blocker. The whole-cell patch clamp recording method was used to measure BK_{Ca} currents after the activation with 30 μM LDD175 and the inhibition with IbTX. Moreover, the enhanced erectile responses shown with the intravenous administration of LDD175 were demonstrated using an in vivo cavernous nerve stimulation study. BK_{Ca} channels are considered as key players in maintaining normal vasomotor tone by regulating K⁺ efflux. Hyperpolarization plays an important role in vascular reactivity by negative feedback of excessive constriction (Ghatta et al., 2006). Besides that, diverse ion channels have been related to the relaxation of smooth muscles. Potassium channels are abundantly expressed in smooth muscles of diverse organs, including penis, and play an important role in the control mechanism of the contraction of smooth muscle cells (Edwards et al., 1991). Adenosine triphosphate (ATP)-sensitive potassium channel (KATP) openers, which have been first developed for overactive bladder, also activated KATP channels. However, these compounds were activated in cardiovascular systems, and brought about unwanted hemodynamic side effects (Fabiyi et al., 2003). Compared with the KATP channels, BK_{Ca} channels are known to be less expressed in cardiovascular tissues (Ghatta et al., 2006). Thus, fewer cardiovascular adverse events might be expected with use of BK_{Ca} channel openers.

Penile erection is mainly mediated by the cyclic guanosine monophosphate (cGMP)/nitric oxide (NO) pathway, and PDE5 inhibitors have been used as first line therapy in ED treatment (Goldstein et al., 1998). The vasodilator effects of NO are partly mediated by the activation of BK_{Ca} channels (Fukami et al., 1998; Lee and Kang, 2001; Gonzalez-Corrochano et al., 2013). The present inventors verified that 10⁻⁵ M LDD175 plus 10⁻⁶M udenafil had more improved relaxation effects than 10⁻⁵ M LDD175 or 10⁻⁶M udenafil only. These results suggest that the action of the mechanism of LDD175 is augmented by PDE5 inhibitors or that the action of the mechanism of PDE5 inhibitors is augmented by LDD175, and thus LDD175 and PDE5 inhibitors had synergistic effects on relaxation. The present inventors, by using cultured human CSM cells, investigated whole-cell K⁺ currents in the presence of the 30 pM NS1619, a typical BK_{Ca} channel opener, in an attempt to compare it with 30 μM LDD175. At a holding potential of +60 mV, peak current densities were induced by NS1619, and inhibited by IbTX. However, at a potential of −40 mV, which corresponds to more similar conditions to normal cell capacitance, LDD175 induced a peak current but current densities did not form with NS1619. The activation threshold of BK_{Ca} channel currents by LDD175 were shifted to more negative voltages, which were close to the resting potential of the cells. This in vitro electrophysiological results showed that LDD175 activated BK_{Ca} channels under more physiological conditions (−40 mV) in contrast to NS1619, and from these results, LDD175 would be expected to more effectively relax CSM compared with NS1619.

In vivo cavernous nerve stimulations were conducted in a series of sub-maximal (control, in the presence of LDD175 and udenafil) and maximal stimulations in each experiment. Sub-maximal stimulation (2 V, 1 Hz, and 60 sec) was introduced to investigate the effects of LDD175 because it would be difficult for LDD175 to induce the additional effect of relaxation compared with control in the setting of maximal stimulation. Maximal stimulation elicited full erection, and then there was no room for further relaxation by LDD175. At the end of each experiment, the full erectile response was repeated to compare it with sub-maximal stimulation in the presence of LDD175, and ensure the cavernous nerve was intact. The ICP/ABP ratio was measured to compare the effects because ICP elevation during the electrical stimulation may be correlated with ABP. In the current study, intravenous administration of LDD175 (10 mg/kg) significantly enhanced the erectile response during electrical stimulation with regards to maximal ICP, maximal ICP/MAP ratio, and the AUC of ICP/MAP ratio. These increases were also induced even with the administration of udenafil (1 mg/kg), and there was no difference between LDD175 and udenafil with respect to the ICP/MAP ratio and the AUC of ICP/MAP ratio.

Drug-related adverse reactions should be of concern in the field of new drug development. Some KATP channels openers (including cromakalim, ZD6169, and WAY-133537) caused significant decreases in arterial pressure at doses effective on the bladder, and these channel openers exhibited low bladder selectivity (Fabiyi et al., 2003). On the other hand, BK_{Ca} channel openers seem to have fewer side effects in the cardiovascular system due to the smaller expression of BK_{Ca} channels in the heart tissue (Ghatta et al., 2006). Pena et al reported an in vivo hemodynamic assessment of LDD175 using Wistar-Kyoto rats (Dela Pena et al., 2009b). In their experiments, the aortic rings of rats were not relaxed by LDD175. In addition, BP and heart rate changes following intravenous administration of the compound were evaluated, showing that 10 mg/kg LDD175 did not cause significant changes in BP and heart rate. The current study has also demonstrated that BP fluctuation and hemodynamical instability were not observed during in vivo cavernous nerve stimulation. However, further experiments and clinical studies need to be done to verify the safety of LDD175.

### CONCLUSION

The present study results validated that LDD175, a BK_{Ca} channel opener, relaxed the erectile tissues of the CSM, which has been pre-contracted by 10⁻⁵ M phenylephrine (PE), in a dose-dependent manner, in vitro. This relaxation was independent from endothelial cells of the corpus cavernosum, and occurred primarily via the BK_{Ca} channel opening. Moreover, the enhanced erectile function of the in vivo animal model using cavernous nerve electrical stimulation tests was verified. These responses are comparable to those seen with udenafil, and LDD175 may also have a synergistic effect with PDE5 inhibitors on the relaxation of cavernous smooth muscles. These results suggest that LDD175 can be a novel ED treatment candidate.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A method for treating or preventing erectile dysfunction, the method comprising:
administering to a subject in need thereof a composition comprising 4-chloro-7-trifluoromethyl-10H-benzo[4,5]furo[3,2-b]indole-1-carboxylic acid or a pharmaceutically acceptable salt thereof.

2. The method of claim 1, wherein the composition activates BK_{Ca} channels of corporal smooth muscle cells.

3. The method of claim 1, wherein the composition promotes the relaxation of corporal smooth muscle.

4. The method of claim 3, wherein the promoting of the relaxation of corporal smooth muscle is independent from endothelial cells of the corpus cavernosum.

5. The method of claim 1, wherein the composition further comprises a pharmaceutically acceptable carrier.

6. The method of claim 1, wherein the pharmaceutically acceptable carrier is selected from the group consisting of lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, and mineral oil.

7. The method of claim 1, wherein the composition is a dosage form selected from the group consisting of a solution, a suspension, an emulsion, an extract, a powder, a granule, a tablet, and a capsule.

8. The method of claim 1, wherein the composition is administered orally or parenterally.

9. The method of claim 8, wherein the parenteral administration is selected from the group consisting of intravenous, subcutaneous, intramuscular, intraperitoneal and transdermal injections.

10. A method for treating or preventing erectile dysfunction, the method comprising:
administering to a subject in need thereof a composition comprising (i) 4-chloro-7-trifluoromethyl-10H-benzo[4,5]furo[3,2-b]indole-1-carboxylic acid and (ii) a phosphodiesterase type 5 (PDE5) inhibitor.

11. The method of claim 10, wherein the PDE5 inhibitor is one selected from the group consisting of avanafil, lodenafil, mirodenafil, sildenafil, tadalafil, vardenafil, udenafil, and zaprinast.

12. The method of claim 11, wherein the PDE5 inhibitor is udenafil.

13. The method of claim 10, wherein the composition further comprises a pharmaceutically acceptable carrier.

14. The method of claim 13, wherein the pharmaceutically acceptable carrier is selected from the group consisting of lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, and mineral oil.

15. The method of claim 10, wherein the composition is a dosage form selected from the group consisting of a solution, a suspension, an emulsion, an extract, a powder, a granule, a tablet, and a capsule.

16. The method of claim 10, wherein the composition is administered orally or parenterally.

17. The method of claim 16, wherein the parenteral administration is selected from the group consisting of intravenous, subcutaneous, intramuscular, intraperitoneal and transdermal injections.
